(19) United States
(12) Patent Application Publication
(10) Pub. No.: US 2024/0001044 A1
(43) Pub. Date: Jan. 4, 2024

(54) SLOPE NEEDLE SET AND SLOPE NEEDLE INCLUDING MICRONEEDLE

(71) Applicant: INCYTO CO., LTD., Cheonan-si, Chungcheongnam-do (KR)

(72) Inventors: Yong Dook BYEON, Cheonan-si, Chungcheongnam-do (KR); Sin Kil CHO, Cheonan-si, Chungcheongnam-do (KR); Hwan Cheol YANG, Cheonan-si, Chungcheongnam-do (KR)

(21) Appl. No.: 18/259,081

(22) PCT Filed: Jan. 4, 2022
(86) PCT No.: PCT/KR2022/000100

§ 371 Date: Jun. 23, 2023

(30) Foreign Application Priority Data
Jan. 5, 2021 (KR) 10-2021-0001088

(51) Int. Cl. A61M 5/32; A61M 5/46; A61M 37/00; A61M 5/34

(52) U.S. Cl. CPC A61M 5/3286; A61M 5/46; A61M 37/0015; A61M 5/345; A61M 2037/0023

(57) ABSTRACT

A slope needle includes a needle base having a predetermined length. The needle base accommodates a cannula therein with a portion of the cannula protruded to an outside; and a slope hub configured to be coupled to a syringe. The slope hub is extended from the needle base. The needle base includes a needle base bevel formed at an end of the needle base from which the cannula protrudes.

### TECHNICAL FIELD

The present disclosure relates to a slope needle set and a slope needle which include a microneedle, and more particularly, to a slope needle set and a slope needle, which include a microneedle, for delivering drugs or functional substances into a skin using the microneedle.

### BACKGROUND

In general, the skin consists of an epidermis disposed on the outside and a dermis disposed on the inside. The epidermis includes a stratum lucidum, stratum corneum, stratum granulosum, and the like, and the dermis includes a subcutaneous fat layer and the like. Various drugs or cosmetics for penetrating into the skin and giving an effective effect may not normally be absorbed by the stratum granulosum of the epidermis.

The epidermis has an average thickness of about 0.15 mm. This epidermis includes a stratum corneum to protect the body from external penetrating. The stratum corneum has a thickness of about 20 μm and plays a key role in the skin protective barrier. Although this protective barrier function is essential to protect the body, it blocks drugs or functional substances even when it is required to deliver drugs or functional substances into the epidermis. Accordingly, in order to deliver drugs or functional substances, methods such as needles and electrophoresis are used.

In addition, control of the depth of a needle is not required in the case of delivering drugs to the entire body, such as intramuscular injection or vascular injection, but for fillers or functional substances that only act on the skin, the effect can be maximized by injecting them into a specific location within the skin.

Accordingly, conventionally, when injecting with a needle, a skilled expert injects substances to a specific depth in the skin, but it is not easy to perform the injection accurately, either, and it is difficult to accurately inject substances into a specific location.

### RELATED ART DOCUMENTS

(Patent document 1) Korean Patent No. 10-1195974, Oct. 24, 2012
(Patent document 2) Korean Patent Application Publication No. 10-2003-0007039, Jan. 23, 2003
(Patent document 3) Korean Patent No. 10-1106286, Jan. 9, 2012

### SUMMARY

Embodiments of the present disclosure are directed to addressing an issue associated with the related art, and to providing a slope needle set and a slope needle, which include a cannula, capable of accurately delivering drugs or functional substances to a desired location using the cannula.

In accordance with a first embodiment of the present disclosure, there is provided a slope needle including: a needle base having a predetermined length, and accommodating a cannula therein with a portion of the cannula protruded to an outside; and a slope hub configured to be coupled to a syringe, the slope hub being extended from the needle base, wherein the needle base includes a needle base bevel formed at an end of the needle base from which the cannula protrudes.

The slope hub may include a hub hole into which one end of the syringe is inserted to be coupled, and the cannula may include a micro hole for injecting one of a drug and a functional substance into a skin of a patient, the micro hole communicating with the hub hole.

The cannula may include an end protruding from the needle base tip bevel, and the end includes a cannula tip bevel.

The cannula tip bevel may be is inclined in the same direction as the needle base tip bevel.

In accordance with a second embodiment of the present disclosure, there is provided a slope needle set including: a slope needle having a predetermined length, and including a cannula therein with a portion of the cannula protruded to an outside; and an adjustment adapter configured to be detachably coupled to the slope needle to adjust a degree to which the cannula protrudes to an outside, wherein the slope needle includes: a needle base having a predetermined length, and including the cannula therein with the portion of the cannula protruded to an outside; and a slope hub configured to be coupled to a syringe, the slope hub being extended from the needle base, and wherein the adjustment adapter includes an adapter tip bevel formed at an end of the adjustment adapter from which the cannula protrudes.

The adjustment adapter may include an adjustment hole formed therein in a longitudinal direction of the adjustment adapter, and the cannula may be exposed to an outside through the adjustment hole.

The needle base may include a needle base tip bevel formed at an end of the needle base from which the cannula protrudes, and the adjustment adapter may be coupled to the slope needle so that the needle base tip bevel and the adapter tip bevel are inclined in the same direction.

The adjustment adapter may be coupled to the slope needle so that a length of the cannula protruding from the adapter tip bevel is shorter than a length of the cannula protruding from the needle base tip bevel.

The end of the needle base may include a surface forming a predetermined angle with the needle base tip bevel.

The end of the needle base may include a plane surface, and an end of the cannula may be disposed on the plane surface of the end of the needle base.

In accordance with a third embodiment of the present disclosure, there is provided a slope needle including: a fixing adapter having a length and including an adapter tip bevel at one end of the fixing adapter; and a cannula coupled to the fixing adapter, wherein the cannula includes: a cannula tip partially protruding from the adapter tip bevel; and a blunt cannula body extending from the cannula tip and having an outer diameter greater than an outer diameter of the cannula tip.

The fixing adapter includes an adapter hole formed therein in a longitudinal direction of the fixing adapter, and the cannula may be inserted into the adapter hole to be coupled to the fixing adapter.

The slope needle may further include a bonding part for fixing the cannula to the adapter hole.

The cannula tip may include a micro hole for injecting one of a drug and a functional substance into a skin of a patient formed therein.

The cannula tip may include a cannula tip bevel at an end protruding from the adapter tip bevel.

The cannula tip bevel may be inclined in the same direction as the adapter tip bevel.

The cannula tip bevel may be inclined at an angle of 10 degrees or more and less than 90 degrees with respect to a longitudinal direction of the cannula.

The cannula tip may be protruded from the adapter tip bevel by a length of 0.07 mm to 3.8 mm in a longitudinal direction of the fixing adapter.

The cannula tip may be protruded from the adapter tip bevel by a length of 0.01 mm to 2 mm in a direction perpendicular to the adapter tip bevel.

An outer diameter of an end of the cannula tip protruding from the adapter tip bevel may range from 0.04 mm to 0.2 mm.

The adapter tip bevel may have a predetermined angle with respect to a longitudinal direction of the fixing adapter.

The predetermined angle may be 10 degrees or more and less than 90 degrees.

The fixing adapter and the cannula may be integrally formed with each other.

In accordance with a fourth embodiment of the present disclosure, there is provided a slope needle set including: a hub coupled to a syringe; and a slope needle coupled to the hub, the slope needle including a cannula disposed therein and protruding to an outside, wherein the slope needle includes: a fixing adapter having a predetermined length to be coupled to the hub and having an adapter tip bevel formed at one end of the fixing adapter; and a cannula including a cannula tip disposed in the slope needle and partially protruding from the adapter tip bevel.

The cannula may further include a blunt cannula body extending from the cannula tip and having an outer diameter greater than an outer diameter of the cannula tip.

The hub may include a hub hole therein, and when the slope needle is coupled to the hub, the blunt cannula body is coupled to the hub hole.

The fixing adapter may include: a cannula coupling portion having the adapter tip bevel formed at one end of the cannula coupling portion; and a hub coupling portion extending from the cannula coupling portion and coupled to the hub.

The blunt cannula body may protrude from the cannula coupling portion toward the hub.

The slope needle may further include a bonding part for fixing the cannula to the fixing adapter.

In one embodiment of the present disclosure, by guiding the slope surface (the inclined surface at a bevel angle) of the slope needle to be inserted in a direction parallel to the surface of the skin, it can be inserted as much as a designed depth, so that drugs or functional substances can be delivered accurately to a desired location.

In addition, in one embodiment of the present disclosure, since the slope needle is coupled to a hub coupled to a syringe so that a slope surface of the cannula and a slope surface of an adapter are parallel, when a user uses the slope needle, by placing the slope surface of the adapter is parallel to the skin, the cannula can be inserted at a desired location, thus allowing convenient injection of drugs or functional substances.

Furthermore, as the needle base end has a shape in which the slope surface of the needle base is partially removed, an operator rotates a syringe by tilting the slope surface of the needle base toward the skin while the needle base end is in contact with the skin of a patient. Hence, it is possible to inject drugs or functional substances into the accurate location.

### BRIEF DESCRIPTION OF THE DRAWINGS

FIG. 1 is a perspective view illustrating a slope needle including a cannula according to a first embodiment of the present disclosure.

FIG. 2 is a cross-sectional view illustrating the slope needle including the cannula according to the first embodiment of the present disclosure.

FIG. 3 is a side view illustrating the slope needle including the cannula according to the first embodiment of the present disclosure.

FIG. 4 is an enlarged view of area A of FIG. 3.

FIG. 5 is an exploded perspective view illustrating a slope needle set including the cannula according to the first embodiment of the present disclosure.

FIG. 6 is a perspective view illustrating the slope needle set including the cannula according to the first embodiment of the present disclosure.

FIG. 7 is a side view illustrating the slope needle set including the cannula according to the first embodiment of the present disclosure.

FIG. 8 is an enlarged view of area B of FIG. 7.

FIG. 9 is a view illustrating a slope needle set including a cannula according to a second embodiment of the present disclosure.

FIG. 10 is an exploded perspective view illustrating the slope needle set including the cannula according to the second embodiment of the present disclosure.

FIG. 11 is a perspective view illustrating the slope needle including the cannula according to the second embodiment of the present disclosure.

FIG. 12 is a view illustrating a cross-section of the slope needle including the cannula according to the second embodiment of the present disclosure.

FIG. 13 is a rear view illustrating the slope needle including the cannula according to the second embodiment of the present disclosure.

FIG. 14 is a front view illustrating the slope needle including the cannula according to the second embodiment of the present disclosure.

FIG. 15 is a side view illustrating the slope needle including the cannula according to the second embodiment of the present disclosure.

FIG. 16 is an enlarged view of area C of FIG. 16.

FIG. 17 is a perspective view illustrating a needle according to the second embodiment of the present disclosure.

FIG. 18 is an enlarged view of area D of FIG. 10.

FIG. 19 is a view illustrating a needle set including a cannula according to a third embodiment of the present disclosure.

FIG. 20 is an exploded perspective view illustrating the needle set including the cannula according to the third embodiment of the present disclosure.

FIG. 21 is a perspective view illustrating a slope needle including the cannula according to the third embodiment of the present disclosure.

FIG. 22 is a view showing a cross-section of the slope needle including the cannula according to the third embodiment of the present disclosure.

FIG. 23 is an enlarged view of area E of FIG. 22.

### DETAILED DESCRIPTION

Hereinafter, specific embodiments for implementing a spirit of the present disclosure will be described in detail with reference to the drawings.

In describing the present disclosure, detailed descriptions of known configurations or functions may be omitted to clarify the present disclosure.

When an element is referred to as being ‘connected’ to, ‘supported’ by, ‘accessed’ to, ‘supplied’ to, ‘transferred’ to, or ‘contacted’ with another element, it should be understood that the element may be directly connected to, supported by, accessed to, supplied to, transferred to, or contacted with another element, but that other elements may exist in the middle.

The terms used in the present disclosure are only used for describing specific embodiments and are not intended to limit the present disclosure. Singular expressions include plural expressions unless the context clearly indicates otherwise.

Further, in the present disclosure, it is to be noted that expressions, such as the upper side and the lower side, are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

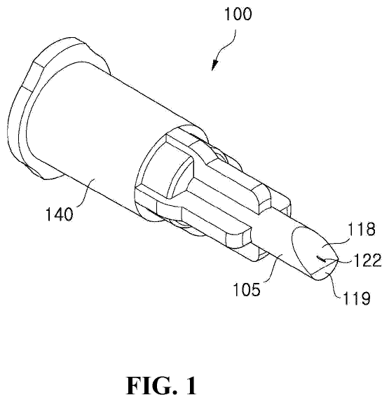

Hereinafter, a slope needle set 10 according to a first embodiment of the present disclosure will be described with reference to FIGS. 1 to 8. The slope needle set 10 according to the first embodiment of the present disclosure is provided for injecting a drug or a functional substance into the skin of a patient.

The needle set 10 according to the first embodiment of the present disclosure includes a slope needle 100, and the slope needle 100 includes a needle base 105 and a slope hub 140.

The needle base 105 includes a cannula 120 to inject a drug or a functional substance into the skin of a patient.

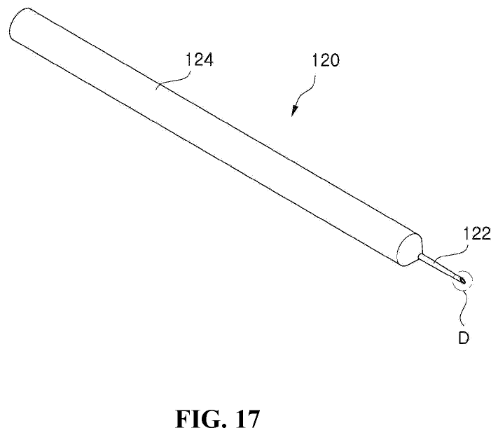

The needle base 105 may be integrally formed with the cannula 120 illustrated in FIG. 17. Here, a needle base tip bevel 118 may be formed at one end of the needle base 105, and an end portion of the cannula 120 may protrude from the needle base tip bevel 118.

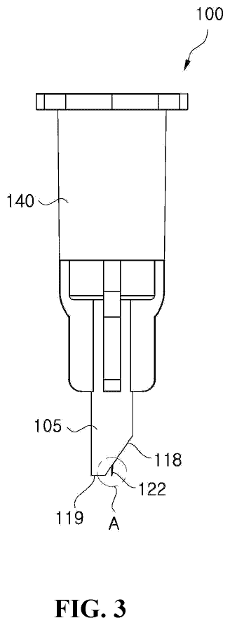

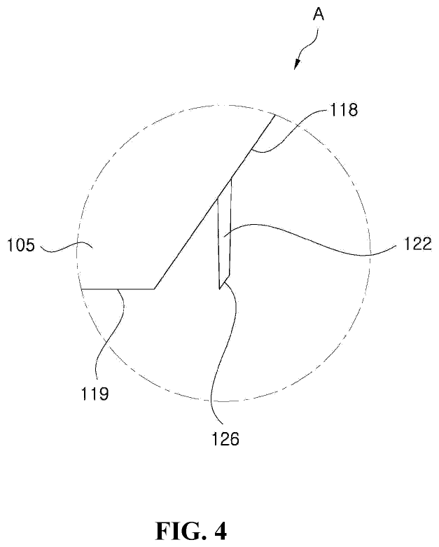

In addition, as illustrated in FIGS. 3 and 4, a needle base end 119 may be formed at the tip of the needle base 105, from which a portion is cut out to form the needle base tip bevel 118. The needle base end 119 may have a planar shape, as in FIG. 4, but is not limited thereto, and may be formed in a curved surface.

The needle base end 119 may be disposed to be substantially flush with the end of the cannula 120. For example, the end of the cannula 120 may protrude by a predetermined length more than the end of the needle base end 119. However, the present disclosure is not limited thereto, and the end of the cannula 120 and the needle base end 119 may be on the same plane, or the end of the cannula 120 may not protrude more than the needle base end 119.

As the end positions of the needle base end 119 and the cannula 120 are positioned at similar planar positions, in a state in which the needle base end 119 is in contact with the skin of a patient, an operator may tilt the needle base tip bevel 118 toward the skin of a patient so that the end of the cannula 120 is inserted into the skin of the patient. In other words, the boundary between the needle base end 119 and the needle base tip bevel 118 may be used as a central axis for tilting (rotating) the needle base 105.

Accordingly, when injecting a drug or a functional substance into the skin of a patient using the needle base 105, the operator can more accurately position the end of the cannula 120.

A hub hole HH is formed in the slope hub 140 so that one end thereof is coupled to an end portion of a conventional syringe, and the needle base 105 is integrally formed at one end of the slope hub 140. For example, as illustrated in FIG. 1, the needle base 105 and the slope hub 140 are integrally formed in a state in which the needle base 105 protrudes from the one end of the slope hub 140 by a predetermined length.

As illustrated in FIG. 1, the slope hub 140 may have an external shape substantially similar to that of a general hub. One end of the slope hub 140 may have a shape in which a portion protrudes in a direction perpendicular to the outer circumferential surface so that other mechanisms may be coupled thereto. In addition, the hub hole HH may be formed at the other end of the slope hub 140 to be coupled to a syringe.

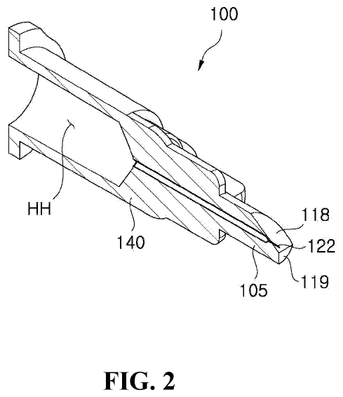

As the cannula 120 is integrally formed with the needle base 105, as illustrated in FIG. 2, a micro hole MH formed in a cannula tip 122 of the cannula 120 may be connected to the hub hole HH formed in the slope hub 140. Here, in the slope needle 100 according to the present embodiment, the needle base 105, the slope hub 140, and the cannula 120 may be integrally formed by injection molding or the like.

As described above, since the slope needle 100 is integrally formed to include the slope hub 140, the slope needle 100 may be directly connected to a general syringe without a connector such as a separate hub.

In addition, as illustrated in FIGS. 3 and 4, the cannula tip 122 is arranged to protrude by a predetermined length from the needle base tip bevel 118 of the needle base 105, and the needle base tip bevel 118 and a cannula tip bevel 126 may be disposed to be inclined in the same direction with each other. For example, the needle base tip bevel 118 and the cannula tip bevel 126 may be disposed to be parallel with each other.

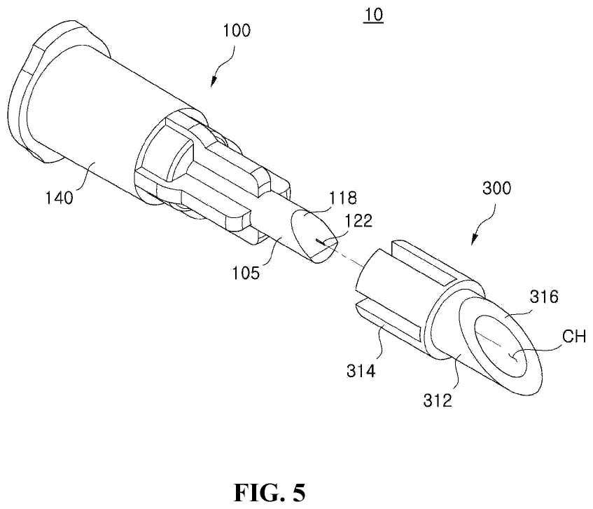

In addition, referring to FIGS. 5 to 8, the needle set 10 includes a slope needle 100 and an adjustment adapter 300.

The adjustment adapter 300 may be used in combination with the slope needle 100, and the length of the cannula tip 122 protruding from the slope needle 100 may be adjusted. The adjustment adapter 300 includes a needle adjustment coupling portion 312 and a hub adjustment coupling portion 314. In addition, the adjustment adapter 300 has a predetermined length, and includes an adjustment hole CH extended in a longitudinal direction of the adjustment adapter 300.

When the adjustment adapter 300 is coupled to the slope needle 100, the needle base 105 may extend through the adjustment hole CH, and the cannula tip 122 may be disposed at one end of the adjustment hole CH.

In addition, an adapter tip bevel 316 may be formed at one end of the needle adjustment coupling portion 312. Accordingly, the adjustment hole CH may be exposed to the adapter tip bevel 316.

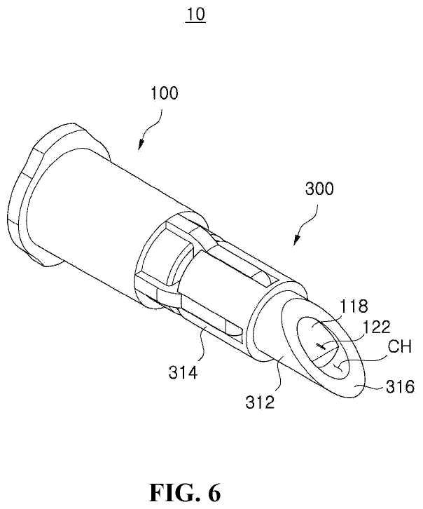

As illustrated in FIGS. 5 and 6, the adjustment adapter 300 may be coupled to the slope needle 100 so that the slope direction of the adapter tip bevel 316 and the slope direction of the needle base tip bevel 118 of the slope needle 100 coincide.

The hub adjustment coupling portion 314 is connected to the needle adjustment coupling portion 312 in a longitudinal direction of the adjustment adapter 300. A plurality of coupling columns may be formed in the hub adjustment coupling portion 314 so that the adjustment adapter 300 is coupled to the slope needle 100. The plurality of coupling columns extends in the longitudinal direction of the adjustment adapter 300 from the needle adjustment coupling portion 312 and may be disposed spaced apart from each other along the circumferential direction. For example, a slit-shaped gap may be formed between the plurality of coupling columns. Each of the plurality of coupling columns of the hub adjustment coupling portion 314 may have an approximate fan shape in cross section.

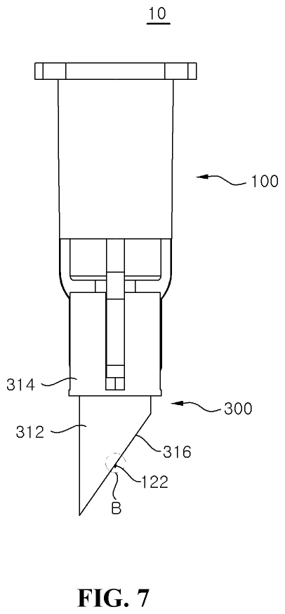

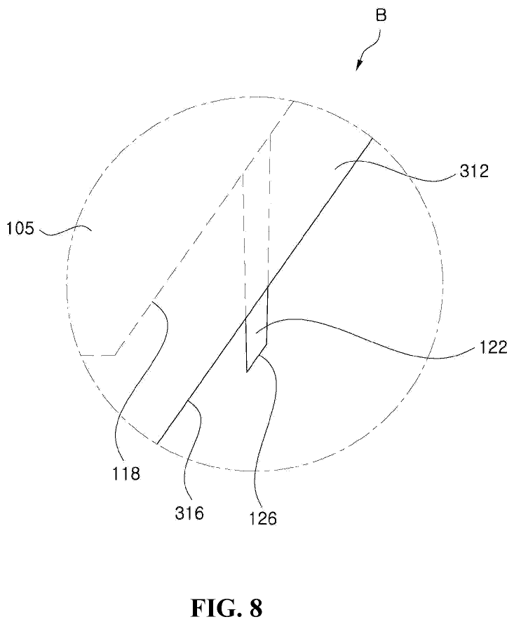

As the hub adjustment coupling portion 314 is formed to have four columns, the adjustment adapter 300 may be coupled to the slope needle 100 according to the present embodiment with directionality. Accordingly, the needle base tip bevel 118 of the slope needle 100 and the adapter tip bevel 316 of the adjustment adapter 300 may be coupled in the same slope direction. When the adjustment adapter 300 is coupled to the slope needle 100 in this way, as illustrated in FIGS. 7 and 8, the length of the cannula tip 122 protruding from the needle base tip bevel 118 of the slope needle 100 may be adjusted by the adjustment adapter 300. For example, when the adjustment adapter 300 is coupled to the slope needle 100, the adapter tip bevel 316 protrudes from the needle base tip bevel 118. Accordingly, the degree to which the cannula tip 122 protruding from the needle base tip bevel 118 protrudes from the adapter tip bevel 316 may be smaller than the protruding degree of the cannula tip 122 from the needle base tip bevel 118.

Here, the degree to which the cannula tip 122 protrudes from the adapter tip bevel 316 may vary depending upon the length of the needle adjustment coupling portion 312 of the adjustment adapter 300. In other words, the adjustment adapters 300 having the needle adjustment coupling portions 312 of different lengths may be provided. Since the adjustment adapters 300 having the needle adjustment coupling portions 312 of different lengths may be coupled to the slope needle 100, the length of the cannula tip 122 protruding from the adapter tip bevel 316 may be adjusted differently.

In addition, when the adjustment adapter 300 is coupled to the slope needle 100, as illustrated in FIG. 8, the needle base tip bevel 118, the adapter tip bevel 316, and the cannula tip bevel 126 may be arranged to have the same inclination angle. For example, the needle base tip bevel 118, the adapter tip bevel 316 and the cannula tip bevel 126 may be parallel to one another.

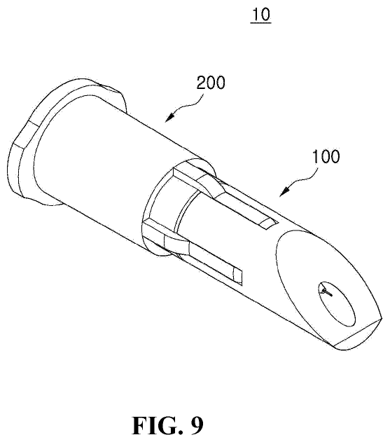

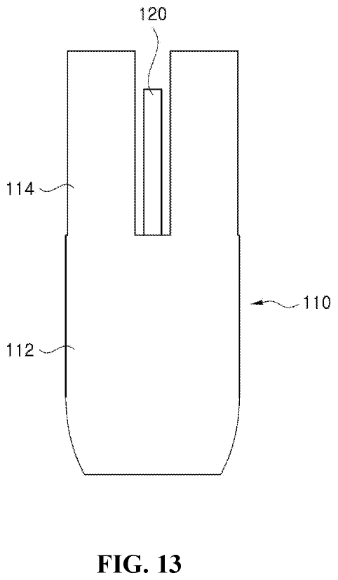

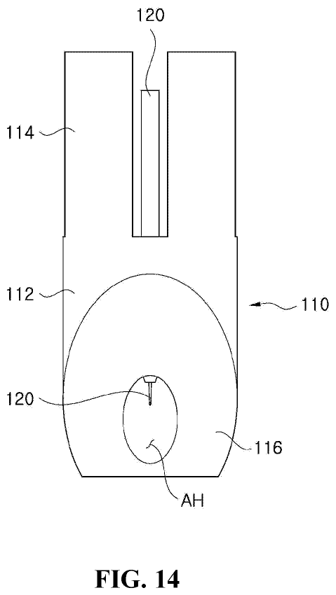

A needle set 10 according to a second embodiment of the present disclosure will be described with reference to FIGS. 9 to 18. The needle set 10 according to the second embodiment of the present disclosure is provided for injecting a drug or a functional substance into the skin of a patient. The needle set 10 includes a slope needle 100 and a hub 200.

The slope needle 100 is coupled to a syringe through the hub 200 and may use a cannula tip 122 to inject a drug or functional substance into the skin of a patient. The slope needle 100 includes a fixing adapter 110, a cannula 120, and a bonding part 130.

The fixing adapter 110 has a predetermined length and may be coupled to the hub 200 coupled to a syringe. In addition, the fixing adapter 110 may have an adapter hole AH into which the cannula 120 is inserted. The fixing adapter 110 includes a cannula coupling portion 112 and a hub coupling portion 114.

The cannula coupling portion 112 may have a substantially cylindrical shape, and an adapter tip bevel 116 may be formed at one end thereof. In addition, the adapter hole AH may be formed in the cannula coupling portion 112. The adapter hole AH may be extended in a longitudinal direction of the cannula coupling portion 112, and may be exposed to the adapter tip bevel 116 formed at the cannula coupling portion 112.

In addition, the end of the cannula coupling portion 112 may have a blunt shape rather than a pointed shape, as illustrated in FIGS. 9 to 15, and if necessary, the tip of the cannula coupling portion 112 may have a planar shape. The adapter tip bevel 116 formed on the cannula coupling portion 112 may have a predetermined angle (θ1) with respect to the longitudinal direction of the cannula coupling portion 112. For example, the angle (θ1) between the adapter tip bevel 116 and the longitudinal direction of the cannula coupling portion 112 may be about 10 degrees to 90 degrees, and may be 10 to 90 degrees exclusive.

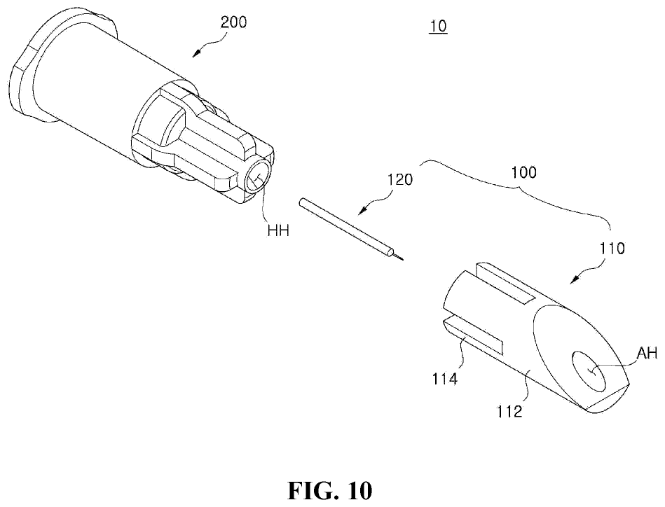

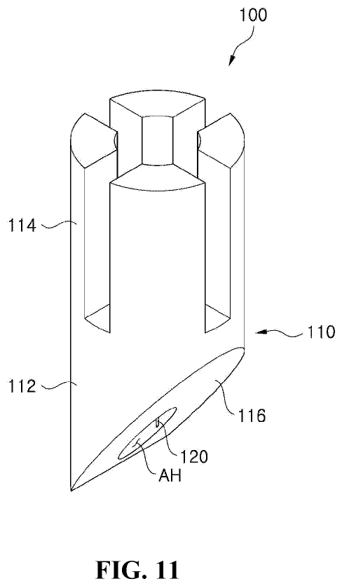

The hub coupling portion 114 is connected to the cannula coupling portion 112 in the longitudinal direction of the fixing adapter 110 from the cannula coupling portion 112. The hub coupling portion 114 may be coupled to the hub 200 coupled to an external syringe. To this end, as illustrated in FIGS. 10 and 11, a plurality of coupling columns may be formed in the hub coupling portion 114. The plurality of coupling columns may extend in the longitudinal direction of the fixing adapter 110 from the cannula coupling portion 112 and may be disposed spaced apart from each other along the circumferential direction. For example, a slit-shaped gap may be formed between the plurality of coupling columns. Each of the plurality of coupling columns may have an approximate fan shape in cross section.

Here, as described above, the shape of the hub coupling portion 114 may be formed to have the plurality of coupling columns to be coupled to the conventional hub 200, but is not limited thereto, and may have various shapes depending on the shape of the hub 200.

The cannula 120 is provided to penetrate the skin and inject drugs or functional substances into the skin. To this end, the cannula 120 includes the cannula tip 122 and a blunt cannula body 124.

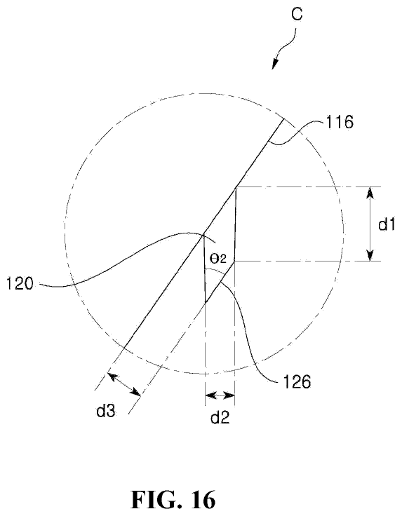

The cannula tip 122 may include an end portion having an outer diameter d2 in micro unit so that the cannula tip 122 is able to penetrate the skin. For example, as illustrated in FIG. 16, the outer diameter d2 of the end portion of the cannula tip 122 may be about 0.40 mm to 0.200 mm.

The cannula tip 122 is provided for insertion into the skin of a patient. The end portion of the cannula tip 122 may have an outer diameter in micro unit, and the cannula tip 122 may have the same outer diameter along the longitudinal direction thereof. However, the present disclosure is not limited thereto, and if necessary, the outer diameter of the cannula tip 122 may increase as goes away from the end in the longitudinal direction of the cannula tip 122.

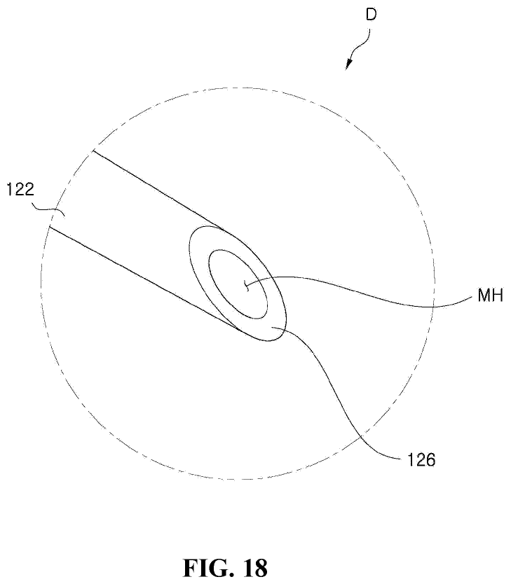

As illustrated in FIG. 18, the end of the cannula tip 122 may have a cannula tip bevel 126 to be inserted into the skin of a patient. As the cannula tip bevel 126 is formed, the end of the cannula tip 122 is formed to be pointed, so that it may be easily inserted into the skin of a patient.

In addition, the cannula tip 122 may have a micro hole MH formed therein. The micro hole MH is formed along the longitudinal direction of the micro needle 122 and may be exposed to the cannula tip bevel 126. Accordingly, the micro hole MH may have an end inclined as the cannula tip bevel 126 in the cannula tip bevel 126.

The blunt cannula body 124 may extend from the other end of the cannula tip 122 to support the cannula tip 122. As illustrated in FIG. 17, the blunt cannula body 124 may be integrally coupled with the cannula tip 122. The blunt cannula body 124 may have an outer diameter greater than that of the cannula tip 122, and may have an outer diameter of several millimeters. In addition, the blunt cannula body 124 may have a length relatively longer than that of the cannula tip 122.

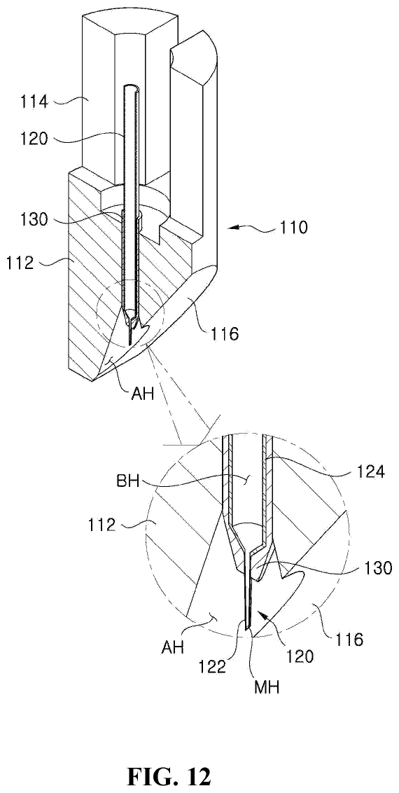

In addition, a blunt hole BH extending from the micro hole MH of the cannula tip 122 may be formed inside the blunt cannula body 124. The width of the blunt hole BH formed in the blunt cannula body 124 may be relatively greater than the width of the micro hole MH, as illustrated in FIG. 12.

Accordingly, the drug or functional substance accommodated in an external syringe may be discharged through the end of the cannula tip 122 via the hub 200 and the blunt hole BH of the blunt cannula body 124 and the micro hole MH of the cannula tip 122.

As described above, the cannula 120 is inserted into the adapter hole AH of the fixing adapter 110, and is fixed to the fixing adapter 110 by the bonding part 130. The cannula 120 may be fixed to the fixing adapter 110 by the bonding part 130 so that the end of the cannula tip 122 partially protrudes from the adapter tip bevel 116 of the fixing adapter 110.

As illustrated in FIG. 12, the bonding part 130 may be applied to the inside of the adapter hole AH of the fixing adapter 110 to fill a space between the outer circumference surface of the cannula 120 and the inner surface of the adapter hole AH. In addition, as illustrated, in some cases, the bonding part 130 may partially protrude to the outside of the adapter hole AH of the fixing adapter 110.

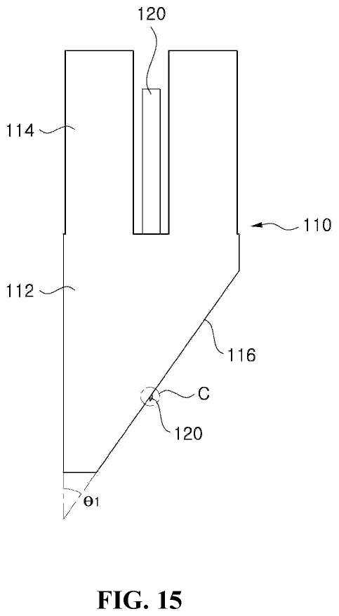

As described above, the cannula 120 may be fixed to the fixing adapter 110 with the end of the cannula tip 122 partially protruding from the adapter tip bevel 116 as illustrated in FIGS. 15 and 16. In this way, the end of the cannula tip 122 may be inserted into the skin of a patient as much as it partially protrudes from the adapter tip bevel 116.

For example, the end of the cannula tip 122 may protrude from the fixing adapter 110 by a length d1 of about 0.07 mm to 3.80 mm in the longitudinal direction of the fixing adapter 110, and a length d3 protruding from the adapter tip bevel 116 of the fixing adapter 110 in a direction perpendicular to the adapter tip bevel 116 may be about 0.01 mm to 2.00 mm.

In addition, the end of the cannula tip 122 may have the cannula tip bevel 126, which may be inclined at an angle (θ2) with respect to the longitudinal direction of the cannula tip 122. The angle (θ2) between the cannula tip bevel 126 and the longitudinal direction of the cannula tip 122 may be, for example, about 10 degrees to 90 degrees, and may be 10 to 90 degrees exclusive. In addition, the cannula tip bevel 126 may be inclined at the same angle as the adapter tip bevel 116 of the fixed adapter 110. For example, the cannula tip bevel 126 may be parallel to the adapter tip bevel 116 of the fixed adapter 110.

When an operator makes the adapter tip bevel 116 of the fixing adapter 110 contact with the skin of a patient parallel thereto, a portion of the cannula tip 122 protruding from the adapter tip bevel 116 may be inserted into the skin of the patient. As the cannula tip 122 protrudes from the adapter tip bevel 116 only enough to pass through the stratum corneum of the skin, a drug or a functional material may be injected into a position just below the stratum corneum.

In addition, as the cannula tip bevel 126 and the adapter tip bevel 116 of the fixing adapter 110 are parallel, a drug or a functional substance may be injected parallel to the lower portion of the stratum corneum of the skin through the micro hole MH exposed to the cannula tip bevel 126.

The hub 200 is coupled to the tip of a conventional syringe, and is provided for coupling with a common needle. The hub 200 may be used for a conventional syringe, and may be combined with the fixing adapter 110 in the present embodiment.

The hub 200 may have a shape capable of being coupled to the hub coupling portion 114 of the fixed adapter 110, and the hub hole HH is formed therein. As the slope needle 100 in which the fixing adapter 110 and the cannula 120 are coupled by the bonding part 130 is coupled to the hub 200, a portion of the blunt cannula body 124 of the cannula 120 may be inserted into the hub hole HH instead of a general needle.

Accordingly, the drug or functional substance accommodated in a syringe may be moved to the blunt cannula body 124 and the cannula tip 122 through the hub 200.

In addition to the above configuration, according to a third embodiment of the present disclosure, another type of the slope needle set 10 may be provided.

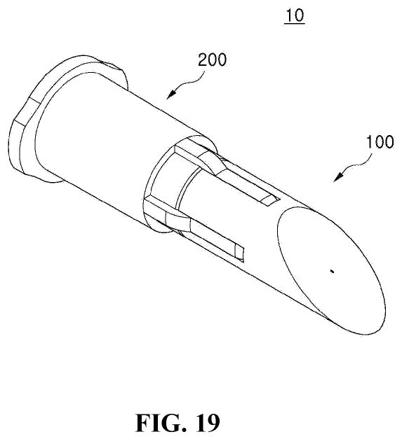

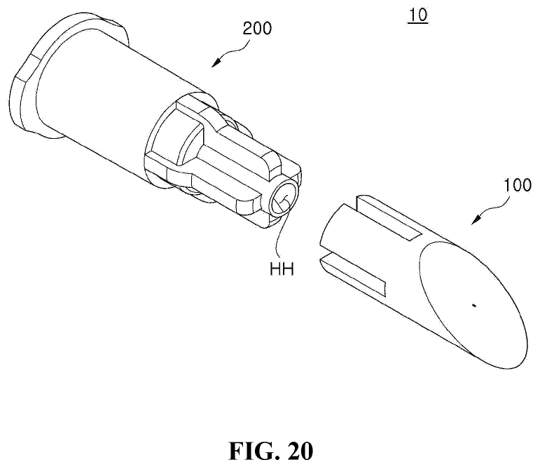

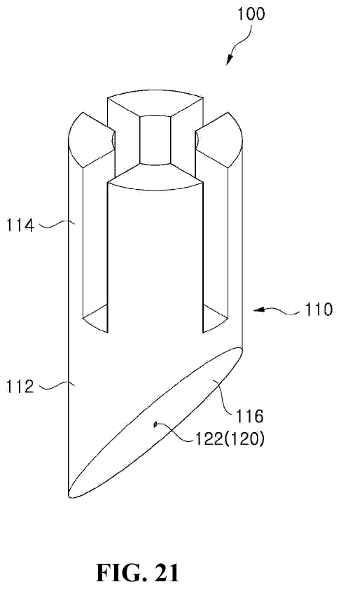

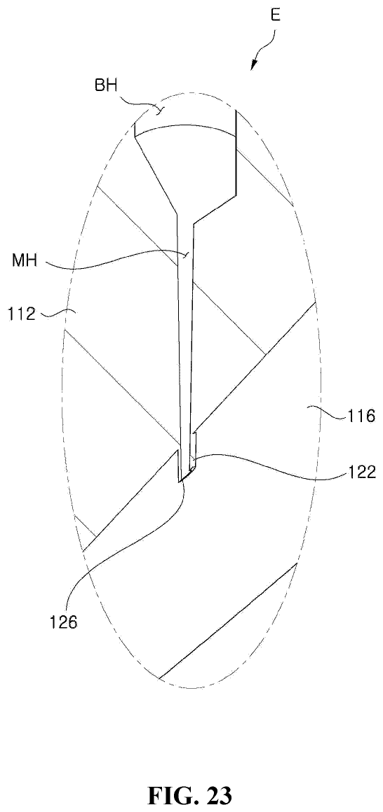

Hereinafter, the third embodiment of the present disclosure will be described with reference to FIGS. 19 to 23. In describing the third embodiment of the present disclosure, the differences between the third embodiment and the above-described embodiments, will be mainly described, and the descriptions in the above-described embodiments will be incorporated for the same description and like reference numerals.

A needle set 10 according to the third embodiment of the present disclosure includes a slope needle 100 and a hub 200.

The slope needle 100 is coupled to a syringe through the hub 200 and uses a cannula tip 122 to inject a drug or functional substance into the skin of a patient.

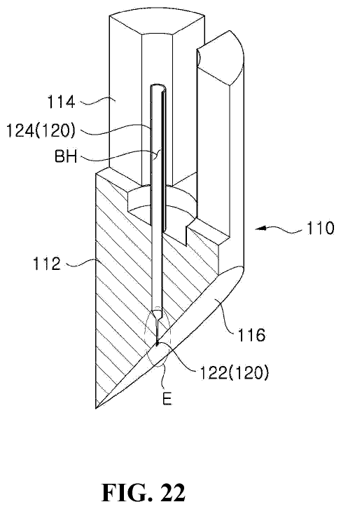

The slope needle 100 includes a fixing adapter 110 and a cannula 120. The fixing adapter 110 may be integrally formed with the cannula 120. As illustrated in FIG. 22, the cannula 120 includes the cannula tip 122 and a blunt cannula body 124, the cannula tip 122 is formed in a state partially protruding from an adapter tip bevel 116 formed at one end of a cannula coupling portion 112 of the fixed adapter 110, and the blunt cannula body 124 may protrude from the other end of the cannula coupling portion 112 of the fixing adapter 110 with a predetermined length in a direction away from the other end of the cannula coupling portion 112.

For example, in the third embodiment, the cannula 120 and the fixing adapter 110 may be integrally formed by injection molding or the like so that the slope needle 100 may have a shape in which the cannula 120 in the second embodiment is inserted into the fixing adapter 110.

Accordingly, the blunt cannula body 124 protrudes from the other end of the cannula coupling portion 112 of the fixing adapter 110 and the hub coupling portion 114 of the fixing adapter 110 is coupled to the hub 200 so that the blunt cannula body 124 is coupled to the hub hole HH.

In addition, the cannula tip 122 may protrude from the adapter tip bevel 116 of the fixed adapter 110 by a predetermined length. The degree to which the cannula tip 122 protrudes from the adapter tip bevel 116 may be the same as in the second embodiment. Further, the cannula tip bevel 126 of the cannula tip 122 may be inclined at the same angle as the adapter tip bevel 116. For example, the cannula tip bevel 126 of the cannula tip 122 may be parallel to the adapter tip bevel 116.

In addition, as described above, since the cannula 120 and the fixing adapter 110 are integrally formed, the adapter hole AH may not be formed in the fixing adapter 110 in the third embodiment.

The examples of the present disclosure have been described above as specific embodiments, but these are only examples, and the present disclosure is not limited thereto, and should be construed as having the widest scope according to the technical spirit disclosed in the present specification. A person skilled in the art may combine/substitute the disclosed embodiments to implement a pattern of a shape that is not disclosed, but it also does not depart from the scope of the present disclosure. In addition, those skilled in the art can easily change or modify the disclosed embodiments based on the present specification, and it is clear that such changes or modifications also belong to the scope of the present disclosure.

1. A slope needle comprising:
a needle base having a predetermined length, and accommodating a cannula therein with a portion of the cannula protruded to an outside; and
a slope hub configured to be coupled to a syringe, the slope hub being extended from the needle base,
wherein the needle base includes a needle base bevel formed at an end of the needle base from which the cannula protrudes.

2. The slope needle of claim 1, wherein the slope hub includes a hub hole into which one end of the syringe is inserted to be coupled, and
wherein the cannula includes a micro hole for injecting one of a drug and a functional substance into a skin of a patient, the micro hole communicating with the hub hole.

3. The slope needle of claim 1, wherein the cannula includes an end protruding from the needle base tip bevel, and the end includes a cannula tip bevel.

4. The slope needle of claim 3, wherein the cannula tip bevel is inclined in the same direction as the needle base tip bevel.

5. A slope needle set comprising:
a slope needle having a predetermined length, and including a cannula therein with a portion of the cannula protruded to an outside; and
an adjustment adapter configured to be detachably coupled to the slope needle to adjust a degree to which the cannula protrudes to an outside,
wherein the slope needle includes:
a needle base having a predetermined length, and including the cannula therein with the portion of the cannula protruded to an outside; and
a slope hub configured to be coupled to a syringe, the slope hub being extended from the needle base, and
wherein the adjustment adapter includes an adapter tip bevel formed at an end of the adjustment adapter from which the cannula protrudes.

6. The slope needle set of claim 5, wherein the adjustment adapter includes an adjustment hole formed therein in a longitudinal direction of the adjustment adapter, and
wherein the cannula is exposed to an outside through the adjustment hole.

7. The slope needle set of claim 5, wherein the needle base includes a needle base tip bevel formed at an end of the needle base from which the cannula protrudes, and
wherein the adjustment adapter is coupled to the slope needle so that the needle base tip bevel and the adapter tip bevel are inclined in the same direction.

8. The slope needle set of claim 7, wherein the adjustment adapter is coupled to the slope needle so that a length of the cannula protruding from the adapter tip bevel is shorter than a length of the cannula protruding from the needle base tip bevel.

9. The slope needle set of claim 1, wherein the end of the needle base includes a surface forming a predetermined angle with the needle base tip bevel.

10. The slope needle set of claim 9, wherein the end of the needle base includes a plane surface, and
wherein an end of the cannula is disposed on the plane surface of the end of the needle base.

11. A slope needle comprising:
a fixing adapter having a length and including an adapter tip bevel at one end of the fixing adapter; and
a cannula coupled to the fixing adapter,
wherein the cannula includes:
a cannula tip partially protruding from the adapter tip bevel; and
a blunt cannula body extending from the cannula tip and having an outer diameter greater than an outer diameter of the cannula tip.

12. The slope needle of claim 11, wherein the fixing adapter includes an adapter hole formed therein in a longitudinal direction of the fixing adapter, and
wherein the cannula is inserted into the adapter hole to be coupled to the fixing adapter.

13. The slope needle of claim 12, further comprising a bonding part for fixing the cannula to the adapter hole.

14. The slope needle of claim 11, wherein the cannula tip includes a micro hole for injecting one of a drug and a functional substance into a skin of a patient.

15. The slope needle of claim 11, wherein the cannula tip includes a cannula tip bevel at an end protruding from the adapter tip bevel.

16. The slope needle of claim 15, wherein the cannula tip bevel is inclined in the same direction as the adapter tip bevel.

17-23. (canceled)

24. A slope needle set comprising:
a hub coupled to a syringe; and
a slope needle coupled to the hub, the slope needle including a cannula disposed therein and protruding to an outside,
wherein the slope needle includes:
a fixing adapter having a predetermined length to be coupled to the hub and having an adapter tip bevel formed at one end of the fixing adapter; and
a cannula including a cannula tip disposed in the slope needle and partially protruding from the adapter tip bevel.

25. The slope needle set of claim 24, wherein the cannula further includes a blunt cannula body extending from the cannula tip and having an outer diameter greater than an outer diameter of the cannula tip, and
wherein the hub includes a hub hole therein, and when the slope needle is coupled to the hub, the blunt cannula body is coupled to the hub hole.

26. (canceled)

27. The slope needle set of claim 25, wherein the fixing adapter includes:
a cannula coupling portion having the adapter tip bevel formed at one end of the cannula coupling portion; and
a hub coupling portion extending from the cannula coupling portion and coupled to the hub.

28. The slope needle set of claim 27, wherein the blunt cannula body protrudes from the cannula coupling portion toward the hub, and
wherein the slope needle further includes a bonding part for fixing the cannula to the fixing adapter.

29. (canceled)
